(19) United States
(12) Patent Application Publication
(10) Pub. No.: US 2013/0043460 A1
(43) Pub. Date: Feb. 21, 2013

(54) CARBAZOLE SERIAL COMPOUNDS

(71) Applicants: Chien-Hong CHENG, Hsin-Chu City (TW); Fang-ly WU, Hsinchu City (TW); Chin-Hsien CHEN, Hsinchu City (TW); Hsiu-Yun TSAI, Hsinchu City (TW); Lun-Chia HSU, Hsinchu City (TW)

(72) Inventors: Chien-Hong CHENG, Hsin-Chu City (TW); Fang-ly WU, Hsinchu City (TW); Chin-Hsien CHEN, Hsinchu City (TW); Hsiu-Yun TSAI, Hsinchu City (TW); Lun-Chia HSU, Hsinchu City (TW)

(21) Appl. No.: 13/316,995

(22) Filed: Dec. 12, 2011

(30) Foreign Application Priority Data
Aug. 17, 2011 (TW) 100129323

(51) Int. Cl. H01L 51/54; C07D 409/10; C07D 401/10; C07D 209/86

(52) U.S. Cl. 257/40; 548/417; 546/173; 257/E51.026

(57) ABSTRACT

Disclosed is a carbazole serial compound, having a general formula as:

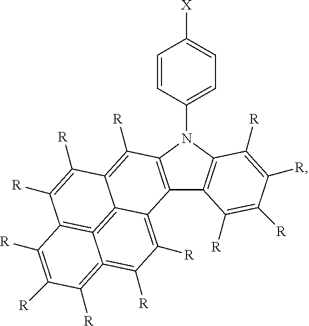

wherein X is selected from a halogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group. Each R is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

### CROSS REFERENCE TO RELATED APPLICATIONS

This application claims priority of Taiwan Patent Application No. 100129323, filed on Aug. 17, 2011, the entirety of which is incorporated by reference herein.

### BACKGROUND OF THE INVENTION

1. Field of the Invention

The disclosure relates to a carbazole serial compound, and in particular relates to an organic light emitting diode utilizing the same.

2. Description of the Related Art

The earliest report of organic electroluminescence was made by Pope et al in 1963, who observed a blue fluorescence from 10-20 μm of crystalline anthracene by applying voltage across opposite sides of a crystal. Thus, the report started a wave of improvements in organic electroluminescence research. However, difficulties of growing large areas of crystals were a challenge. The driving voltage of the devices at the time were too high and the efficiency of organic materials was lower than that of inorganic materials. Because of the disadvantages, the devices were not widely applied due to practical purposes.

However, a major development in organic electroluminescence technology was reported in 1987. Tang and VanSlyke of Eastman Kodak Company used vacuum vapor deposition and novel hetero junction techniques to prepare a multilayered device with hole/electron transporting layers. 4,4-(cyclohexane-1,1-diyl)bis(N,N-dip-tolylbenzenamine) (TPAC) was used as the hole transporting layer, and Alq₃ (tris(8-hydroxyquinolinato) aluminum(III)) film with good film-forming properties was used as an electron transporting and emitting layer. A 60-70 nm-thick film was deposited by vacuum vapor deposition with a low-work function Mg:Ag alloy as a cathode for efficient electron and hole injections. The bi-organic-layer structure allowed the holes and electrons to recombine at the p-n interface and then emit light. The device emitted green light of 520 nm, and was characterized by low driving voltage (<10 V), high quantum efficiency (>1%) and good stability. The improvements, once again, arouse interest in organic electroluminescence research.

Meanwhile, Calvendisg and Burroughes et al. at Cambridge University in 1990 were the first to report using conjugated polymer PPV (poly(phenylene vinylene)) as an emitting layer in a single-layered device structure by solution spin coating. The development of an emitting layer with conjugated polymer drew great interest and quickly sparked research due to the simplicity of fabrication, good mechanical properties of polymer, and semiconductor-like properties thereof. In addition, a large number of organic polymers are known to have high fluorescence efficiencies.

In Japan Patent Publication No. P2010-73987, many carbazole serial compounds are disclosed without discussing their applications, such as in OLEDs. In addition, device performances corresponding to substituent groups in different positions of the carbazole serial compounds are not disclosed.

### BRIEF SUMMARY OF THE INVENTION

One embodiment of the disclosure provides a carbazole serial compound, having a formula:

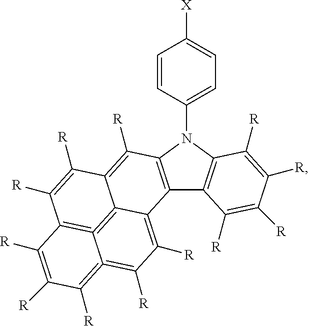

wherein X is selected from a halogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group, and each R is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

One embodiment of the disclosure provides a carbazole serial compound, having a formula:

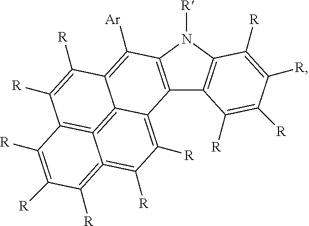

wherein Ar is selected from a para-t-butyl phenyl group, a biphenyl group, a naphthalenyl group, or a thienyl group; wherein R′ is selected from a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, or a substituted or non-substituted C₄₋₄₀ hetero aryl group; and wherein each R is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

One embodiment of the disclosure provides an organic light emitting diode, comprising: an anode; a cathode; and an organic layer disposed between the anode and the cathode, wherein the organic layer comprises the described carbazole serial compound.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 shows photoluminescence intensities of solutions of carbazole serial compounds in several examples of the disclosure.

### DETAILED DESCRIPTION OF THE INVENTION

The following description is of the best-contemplated mode of carrying out the disclosure. This description is made for the purpose of illustrating the general principles of the disclosure and should not be taken in a limiting sense. The scope of the disclosure is best determined by reference to the appended claims.

The disclosure provides a carbazole serial compound serving as a host material or a guest material in a light emitting layer of an organic light emitting diode (OLED). Because the carbazole serial compound has excellent thermal stability and luminescence efficiency, it may further enhance the lifetime and brightness of an OLED.

The described carbazole compound can be synthesized as shown in Formulae 1 and 2:

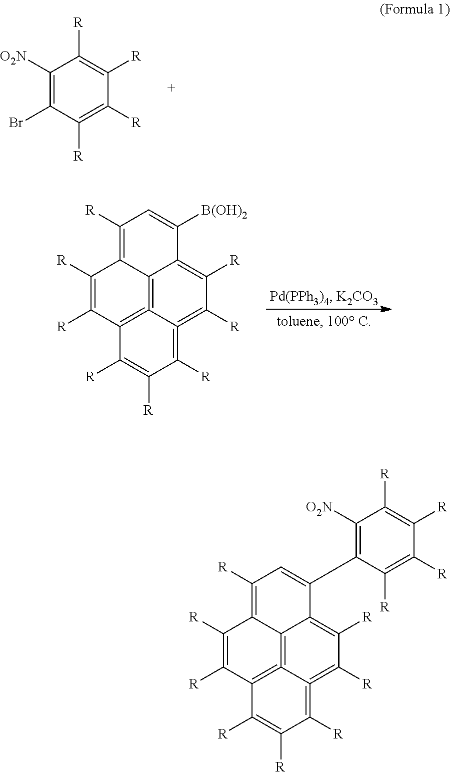

The reaction in Formula 1 is the so-called Suzuki coupling. Each R of the benzene ring and the pyrene ring is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group. The product of the Suzuki coupling in Formula 1 is then processed according to a cyclization (Cadogan reaction) as shown in Formula 2.

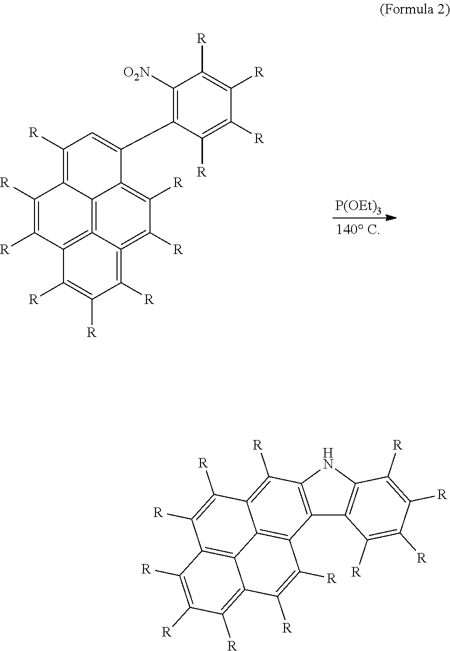

In one embodiment, the product of Formula 2 is directly processed a Buchwald-Hartwig coupling as shown in Formula 3. In Formula 3, X is selected from a halogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

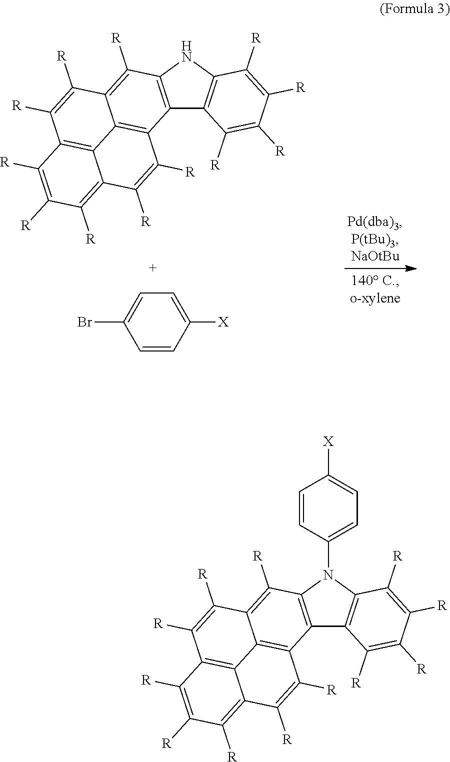

In another embodiment, the product of Formula 2 is firstly processed a substitution reaction, as shown in Formula 4. R′ is selected from a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, or a substituted or non-substituted C₄₋₄₀ hetero aryl group. If R′ is an aryl or a hetero aryl group, the substitution reaction in Formula 4 would not work and the Buchwald-Hartwig coupling in Formula 3 would be adopted.

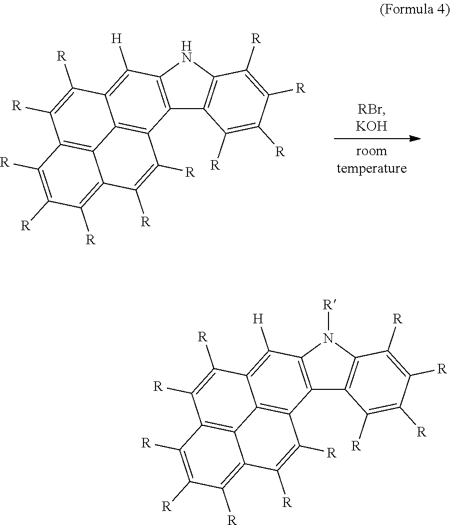

Subsequently, the product of Formula 4 serves as a starting material in a bromination reaction, as shown in Formula 5. It should be illustrated that if all Rs are hydrogen atoms, the position symbolized “H” on the starting material in Formula 5 will be firstly brominated. It should be understood that the bromination position “H” of the starting material in Formula 5 must be a hydrogen atom, and the other R can be hydrogen or other substituent groups such as a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

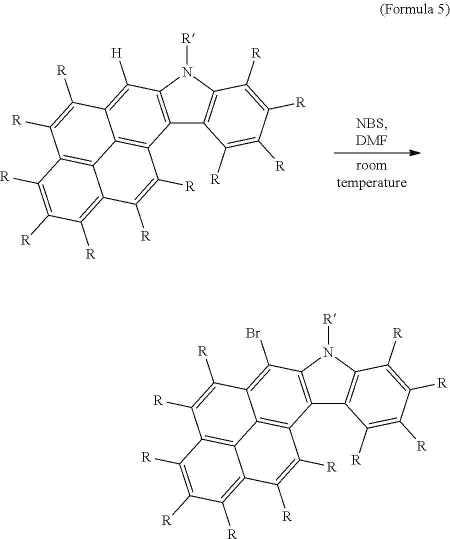

Subsequently, the product of Formula 5 is processed a Suzuki coupling, as shown in Formula 6. In Formula 6, Ar is selected from a para-t-butyl phenyl group, a biphenyl group, a naphthalenyl group, or a thienyl group

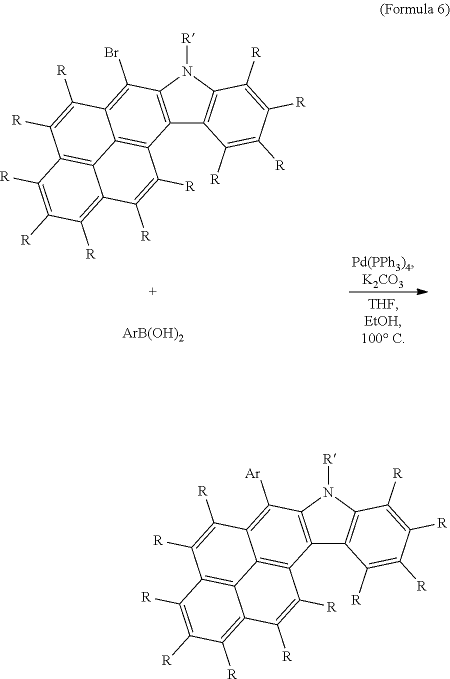

The disclosure further provides an organic light emitting diode (OLED), including an anode, a cathode, and a light emitting layer disposed between the anode and the cathode, wherein the light emitting layer includes the described carbazole serial compound. The anode includes indium tin oxide, indium zinc oxide, aluminum zinc oxide, or combinations thereof. The anode can be formed by evaporation or sputtering. The cathode includes inorganic conductive material such as magnesium silver alloy, lithium fluoride, aluminum, or combinations thereof. The cathode can be formed by evaporation or sputtering. In one embodiment, a hole injecting layer, a hole transporting layer, and/or other suitable layered materials can be disposed between the light emitting layer and the anode. The hole injecting layer includes molybdenum trioxide, copper phthalocyanine, poly(3,4-ethylenedioxythiophene):poly(styrenesulfonate) (PEDOT:PSS), and 4,4′,4″-tris(N-3-methylphenyl-N-phenyl-amino)-triphenylamine (m-TDATA). The hole transporting layer includes 4′4′,4″-tris(carbazol-9-yl)-triphenylamine (TCTA), N,N′-diphenyl-N,N′-bis(3-methylphenyl)-1,1′-biphenyl)-4,4′-diamine (TPD), or N,N′-bis(1-naphyl)-N,N′-diphenyl-1,1′-biphenyl-4,4′-diamine (NPB).

In one embodiment, an electron injecting layer, an electron transporting layer, a hole blocking layer, and/or other suitable layered materials can be disposed between the light emitting layer and the cathode. The electron injecting layer includes alkali halide, alkaline-earth halide, alkali oxide, or alkali carbonate, such as LiF, CsF, NaF, CaF₂, Li₂O, Cs₂O, Na₂O, Li₂CO₃, Cs₂CO₃, or Na₂CO₃. The electron transporting layer includes tris(8-hydroxy quinoline) aluminum (Alq3) or 2,2′,2″-(1,3,5-benzinetriyl)-tris(1-phenyl-1-H-benzimidazole) (TPBI). The hole blocking layer includes 2,9-dimethyl-4,7-diphenyl-1,10-phenanthroline (BCP), aluminum (III) bis(2-methyl-8-quninolinato)-4-phenylphenolate (BAlq), or 2,2′,2″-(1,3,5-Benzinetriyl)-tris(1-phenyl-1-H-benzimidazole) (TPBI).

When the carbazole serial compound serves as the host material of the light emitting layer, the light emitting layer may be further doped with other dopants such as BCzVBi as shown in Formula 7. As such, the luminescence efficiency of the OLED is enhanced by the host-guest system.

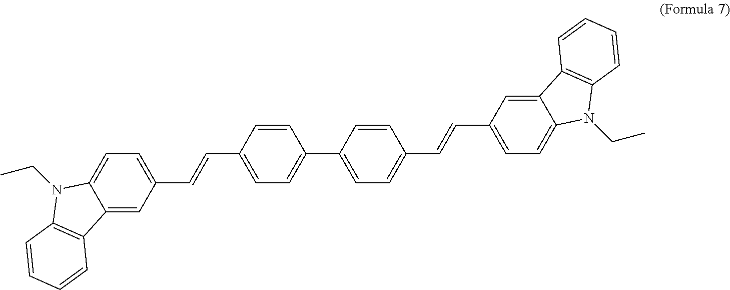 [SMILES: CCn1c2ccccc2c2cc(/C=C/c3ccc(-c4ccc(/C=C/c5ccc6c(c5)c5ccccc5n6CC)cc4)cc3)ccc21]

When the carbazole serial compound serves as the guest material (dopant) of the light emitting layer, the host material of the light emitting layer can be 1,1′-(2,5-dimethyl-1,4-phenylene)dipyrene (DMPPP) as shown in Formula 8. As such, the luminescence efficiency of the OLED is enhanced by the host-guest system.

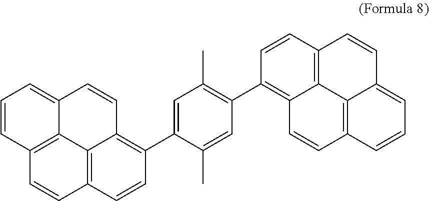 [SMILES: Cc1cc(-c2ccc3ccc4cccc5ccc2c3c45)c(C)cc1-c1ccc2ccc3cccc4ccc1c2c34]

In another embodiment, other conventional host materials and guest materials (dopant) serve as the light emitting layer of the OLED, and the described carbazole serial compounds serve as the hole transporting layer, the electron injecting layer, the electron transporting layer, the hole blocking layer, the hole injecting layer, and/or another organic layer between the anode and the cathode if necessary.

### EXAMPLES

### Preparation Example 1

3.35 g of 1-bromo-2-nitrobenzene (16.58 mmole), 6.12 g of pyren-1-ylboronic acid (24.88 mmole), and 22.11 g of potassium carbonate (15.99 mmole) were charged in a two-necked bottle. 80 mL of water, 240 mL of toluene, and drops of quaternary ammonium salt (aliquat 336) were added into the on-necked bottle. The mixture was heated to 60° C. to dissolve the solid, and the bottle was then vacuumed and purged with nitrogen several times. 0.50 g of Pd(PPh₃)₄ (0.43 mmole) was rapidly added into the bottle, and the bottle was then vacuumed and purged with nitrogen several times. The reaction under nitrogen was heated to 100° C. and reacted at 100° C. for two days. The resulting was cooled to room temperature to obtain a crude. The crude was extracted by ethyl acetate and water to collect an organic layer thereof. The organic layer was dried by MgSO₄, and silica gel was then added to the organic layer. The organic layer was then condensed to remove the solvent thereof, and the extracted crude was dry loaded on the silica gel. The extracted crude was purified by chromatography with an eluent of n-hexane and ethyl acetate (7:1) to obtain a yellow solid (yield=92.93%). The above reaction is shown as Formula 9. The spectra data of the product of Formula 9 is shown as follows. ¹H NMR (400 MHz, CDCl₃): 7.56-7.76 (m, 4H), 7.85 (d, 1H, J=8.0 Hz), 7.98-8.03 (m, 2H), 8.09-8.22 (m, 6H). ¹³C NMR (100 M Hz, CDCl₃): 123.9, 124.2, 124.5, 124.6, 124.7, 125.2, 125.5, 126.1, 126.3, 127.2, 127.8, 128.2, 128.6, 128.7, 138.7, 131.1, 131.3, 132.4, 132.5, 133.4, 135.6, 149.9. HRMS (EI, m/z): calcd for C₂₂H₁₃NO₂ 323.0946. found 323.0945 (M⁺).

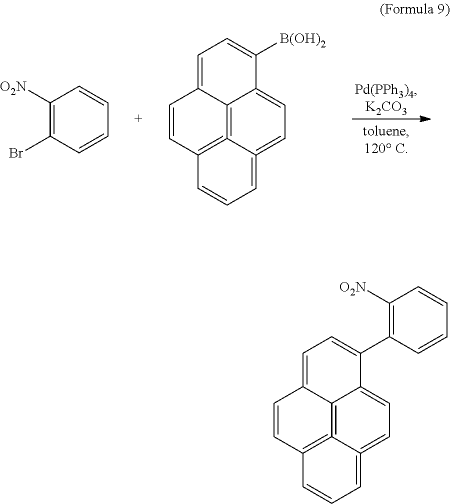

### Preparation Example 2

4.60 g of the product of Formula 9 (14.23 mmole) was charged in a one-necked bottle. 10.32 mL of P(OEt)₃ (56.92 mmole) was then added to the one-necked bottle. The bottle was then vacuumed and purged with nitrogen several times. The mixture under nitrogen was heated to 140° C. and reacted at 140° C. for 24 hours. The resulting was vacuum distillated to remove a major part of the solvent to obtain a crude. Ethyl acetate and silica gel were added to the crude and stirred for 30 minutes. The ethyl acetate was removed by a rotary condenser to dry load the crude on the silica gel. The crude was purified by chromatography with an eluent of n-hexane and ethyl acetate (7:1) to obtain a yellow solid (yield=68.13%). The above reaction is shown as Formula 10. The spectra data of the product of Formula 10 is shown as follows. ¹H NMR (400 M Hz, CDCl₃): 7.46 (dd, 1H, J=7.2, 8.0 Hz), 7.56 (dd, 1H, J=7.2, 8.0 Hz), 7.64 (d, 1H, J=8.0 Hz), 7.97 (dd, 1H, J=7.6, 8.0 Hz), 8.04 (dd, 1H, J=7.6 Hz), 8.10 (d, 1H, J=8.8 Hz), 8.18-8.20 (m, 2H), 8.26 (d, 1H, J=8.0 Hz), 8.36 (d, 1H, J=8.8 Hz), 8.58 (bs, 1H), 8.80 (d, 1H, J=8.0 Hz), 9.13 (d, 1H, J=8.8 Hz). ¹³C NMR (100 M Hz, d-Acetone): 108.7, 112.0, 117.6, 120.4, 120.5, 123.4, 124.2, 124.4, 125.5, 125.8, 126.1, 126.3, 126.4, 127.3, 127.7 128.7, 129.0, 130.8, 131.1, 131.4 139.7, 141.9. HRMS (EI, m/z): calcd for C₂₂H₁₃N, 291.1048. found 291.1047 (M⁺).

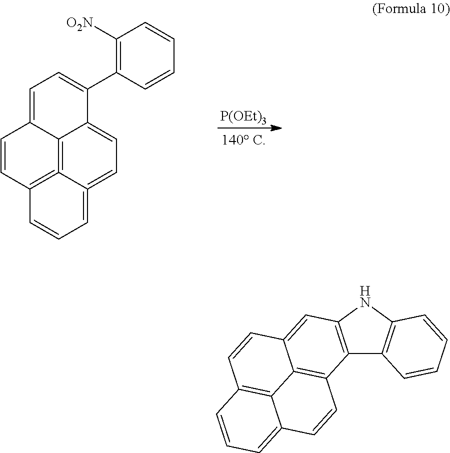

### Preparation Example 3

0.29 g of the product of Formula 10 (1 mmole) and 0.34 g of potassium hydroxide (6.00 mmole) were dissolved in 15 mL of acetone. 0.45 mL of bromoethane (6.00 mmole) was slowly and dropwise added to the acetone solution, and the mixture was reacted at room temperature overnight. The resulting was poured into ice water to precipitate a solid and then filtered. The filtered cake was washed by methanol to obtain a yellow brown solid (yield=93.56%). The above reaction is shown as Formula 11. The spectra data of the product of Formula 11 is shown as follows. ¹H NMR (400 M Hz, CDCl₃): 1.53-1.64 (m, 3H), 4.66 (q, 2H), 7.44-7.48 (m, 1H), 7.59-7.65 (m, 2H), 7.96 (dd, 1H, J=8.0, 7.6 Hz), 8.05 (d, 1H, 8.8 Hz), 8.14-8.20 (m, 3 Hz), 8.25 (d, 1H, 8.0 Hz), 8.34 (d, 1H, 9.2 Hz), 8.72 (d, 1H, 7.6 Hz), 9.15 (d, 1H, 9.2 Hz). ¹³C NMR (100 M Hz, CDCl₃): 13.8, 37.6, 105.0, 108.5, 116.7, 119.3, 119.6, 122.8, 123.3, 123.4, 124.4, 125.0, 125.2, 125.3, 125.4, 126.7, 126.9, 127.7, 128.1, 129.7, 130.0, 130.3, 138.4, 140.5. HRMS (EI, m/z): calcd for C₂₄H₁₇N, 319.1361. found 319.1358 (M⁺).

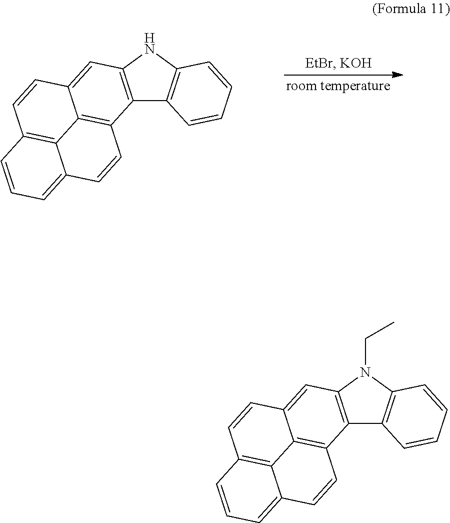 [SMILES: c1cc2ccc3cc4[nH]c5ccccc5c4c4ccc(c1)c2c34 ; CCn1c2ccccc2c2c3ccc4cccc5ccc(cc21)c3c54]

### Preparation 4

0.61 g of N-bromosuccinimide (3.43 mmole) was dissolved in 10 mL of dimethyl formamide, and 0.91 g of the product of Formula 11 was dissolved in 40 mL of dimethyl formamide, respectively. The NBS solution was slowly and dropwise added to the solution of the product of Formula 11 at room temperature. The mixture was then reacted at room temperature for 3 hours. The resulting was poured into water to precipitate a solid and then filtered. The filtered cake was washed by a small amount of methanol to obtain a yellow green solid (yield=93.33%). The above reaction is shown as Formula 12. The spectra data of the product of Formula 12 is shown as follows. ¹H NMR (400 M Hz, CDCl₃): 1.54-1.64 (m, 3H), 5.16 (q, 2H), 7.45-7.50 (m, 1H), 7.63-7.68 (m, 2H), 7.98 (dd, 1H, J=7.6, 7.6 Hz), 8.14 (d, 1H, 6.8 Hz), 8.21 (d, 1H, 7.6 Hz), 8.24 (d, 1H, 7.6 Hz), 8.31 (d, 1H, 9.2 Hz), 8.72 (d, 1H, 9.2 Hz), 8.82 (d, 1H, 8.0 Hz), 9.14 (d, 1H, 9.2 Hz). ¹³C NMR (100 M Hz, CDCl₃): 15.8, 39.9, 102.0, 109.3, 119.4, 120.3, 120.7, 122.8, 122.9, 123.1, 124.5, 125.0, 125.4, 125.8, 126.0, 126.1, 128.4, 128.5, 128.9, 129.8, 130.0, 135.8, 142.2. HRMS (EI, m/z): calcd for C₂₄H₁₆BrN 397.0466. found 397.0463 (M⁺).

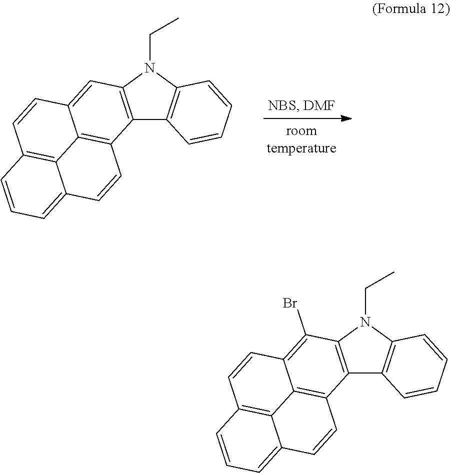 [SMILES: CCn1c2ccccc2c2c3ccc4cccc5ccc(cc21)c3c54 ; CCn1c2ccccc2c2c3ccc4cccc5ccc(c(Br)c21)c3c54]

### Example 1

0.40 g of the product of Formula 12 (1.00 mmole), 0.36 g of 4-tert-butylphenylboronic acid (2.00 mmole), and 3.32 g of potassium carbonate (24.00 mmole) were charged in a one-necked bottle. 12 mL of water, 50 mL of tetrahydrofuran (THF), and 10 mL of ethanol were added into the one-necked bottle. The mixture was heated to 60° C. to dissolve the solid, and the bottle was then vacuumed and purged with nitrogen several times. 0.06 g of Pd(PPh₃)₄ (1.00 mmole) was rapidly added into the bottle, and the bottle was then vacuumed and purged with nitrogen several times. The reaction under nitrogen was heated to 100° C. and reacted at 100° C. for two days. The resulting was cooled to room temperature to obtain a crude. The crude was extracted by ethyl acetate and water to collect an organic layer thereof. The organic layer was dried by MgSO₄, and silica gel was then added to the organic layer. The organic layer was then condensed to remove the solvent thereof, and the extracted crude was dry loaded on the silica gel. The extracted crude was purified by chromatography with an eluent of n-hexane to obtain a yellow solid (yield=44.32%). The above reaction is shown as Formula 13. The spectra data of the product of Formula 13 is shown as follows. ¹H NMR (400 M Hz, CDCl₃): 1.04 (t, 3H), 1.47 (s, 9H), 3.95 (q, 2H), 7.45-763 (m, 7H), 7.73 (d, 1H, 9.2 Hz), 7.88 (d, 1H, 9.2 Hz), 7.95 (dd, 1H, 7.6 Hz), 8.12 (d, 1H, 7.2 Hz), 8.24 (d, 1H, 7.6 Hz), 8.35 (d, 1H, 9.2 Hz), 8.90 (d, 1H, 8.0 Hz), 9.26 (d, 1H, 9.2 Hz). ¹³C NMR (100 M Hz, CDCl₃): 14.2, 31.6, 34.8, 39.0, 109.2, 117.6, 119.7, 121.1, 123.0, 123.4, 124.6, 124.9, 125.2, 125.3, 125.5, 125.7, 126.3, 126.6, 128.1, 129.3, 130.1, 130.2, 130.9, 141.8, 151.2. HRMS (EI, m/z): calcd for C₃₄H₂₉N, 451.2300. found 451.2302 (M⁺). Anal. Calcd. for C₃₄H₂₉N: C, 90.43; H, 6.47; N, 3.10%. Found: C, 90.46; H, 6.47; N, 2.96%.

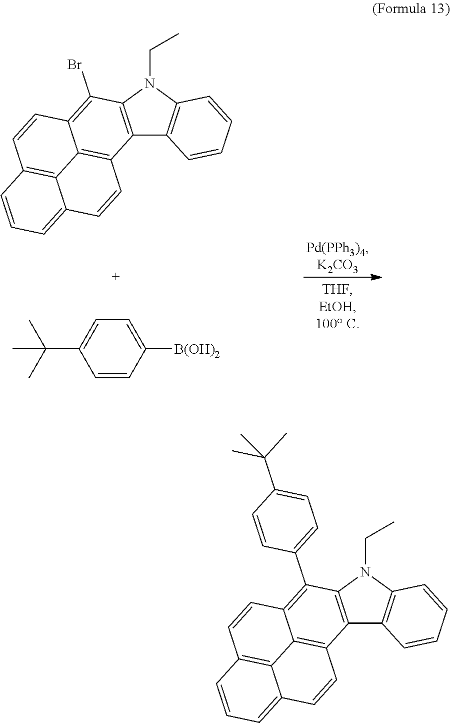 [SMILES: CCn1c2ccccc2c2c3ccc4cccc5ccc(c(Br)c21)c3c54 ; CC(C)(C)c1ccc(B(O)O)cc1 ; CCn1c2ccccc2c2c3ccc4cccc5ccc(c(-c6ccc(C(C)(C)C)cc6)c21)c3c54]

### Example 2

0.40 g of the product of Formula 12 (1.00 mmole), 0.34 g of naphthalen-2-ylboronic acid (2.00 mmole), and 3.32 g of potassium carbonate (24.00 mmole) were charged in a one-necked bottle. 12 mL of water, 50 mL of THF, and 10 mL of ethanol were added into the one-necked bottle. The mixture was heated to 60° C. to dissolve the solid, and the bottle was then vacuumed and purged with nitrogen several times. 0.06 g of Pd(PPh₃)₄ (1.00 mmole) was rapidly added into the bottle, and the bottle was then vacuumed and purged with nitrogen several times. The reaction under nitrogen was heated to 100° C. and reacted at 100° C. for two days. The resulting was cooled to room temperature to obtain a crude. The crude was extracted by ethyl acetate and water to collect an organic layer thereof. The organic layer was dried by MgSO₄, and silica gel was then added to the organic layer. The organic layer was then condensed to remove the solvent thereof, and the extracted crude was dry loaded on the silica gel. The extracted crude was purified by chromatography with an eluent of n-hexane to obtain a yellow solid (yield=44.92%). The above reaction is shown as Formula 14. The spectra data of the product of Formula 14 is shown as follows. ¹H NMR (400 M Hz, CDCl₃): 1.00 (t, 3H), 3.84-4.00 (m, 2H), 7.46-7.74 (m, 7H), 7.84 (d, 1H, 9.2 Hz), 7.90-7.98 (m, 2H), 8.00-8.14 (m, 4H), 8.26 (d, 1H, 7.6 Hz), 8.37 (d, 1H, 9.2 Hz), 8.93 (d, 1H, 9.2 Hz), 9.28 (d, 1H, 9.2 Hz). ¹³C NMR (100 M Hz, CDCl₃): 14.0, 38.9, 124.7, 124.8, 125.1, 125.3, 125.4, 125.5, 126.4, 126.6, 126.7, 126.8, 126.9 127.0, 127.9, 127.9, 128.0, 128.1, 128.1, 128.2. HRMS (EI, m/z): calcd for C₃₄H₂₃N, 445.1830. found 445.1831 (M⁺).

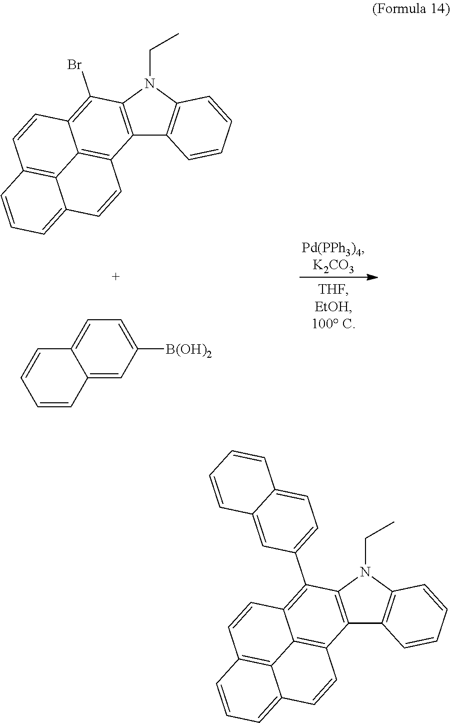 [SMILES: CCn1c2ccccc2c2c3ccc4cccc5ccc(c(Br)c21)c3c54 ; OB(O)c1ccc2ccccc2c1 ; CCn1c2ccccc2c2c3ccc4cccc5ccc(c(-c6ccc7ccccc7c6)c21)c3c54]

### Example 3

0.4 g of the product of Formula 12 (1.00 mmole), 0.39 g of biphenyl-4-ylboronic acid (2.00 mmole), and 3.32 g of potassium carbonate (24.00 mmole) were charged in a one-necked bottle. 12 mL of water, 50 mL of THF, and 10 mL of ethanol were added into the one-necked bottle. The mixture was heated to 60° C. to dissolve the solid, and the bottle was then vacuumed and purged with nitrogen several times. 0.06 g of Pd(PPh₃)₄ (1.00 mmole) was rapidly added into the bottle, and the bottle was then vacuumed and purged with nitrogen several times. The reaction under nitrogen was heated to 100° C. and reacted at 100° C. for two days. The resulting was cooled to room temperature to obtain a crude. The crude was extracted by ethyl acetate and water to collect an organic layer thereof. The organic layer was dried by MgSO₄, and silica gel was then added to the organic layer. The organic layer was then condensed to remove the solvent thereof, and the extracted crude was dry loaded on the silica gel. The extracted crude was purified by chromatography with an eluent of n-hexane to obtain a yellow solid (yield=42.44%). The above reaction is shown as Formula 15. The spectra data of the product of Formula 14 is shown as follows. ¹H NMR (400 M Hz, CDCl₃): 1.08 (t, 3H), 4.04 (q, 2H), 7.41 (m, 5H), 7.68 (d, 2H, J=8.0 Hz), 7.75-7.82 (m, 4H), 7.86-7.93 (m, 3H), 7.96 (dd, 1H, J=7.6, 7.2 Hz), 8.14 (d, 1H, J=7.6 Hz), 8.26 (d, 1H, J=7.2 Hz), 8.37 (d, 1H, J=8.8 Hz), 8.92 (d, 1H, J=7.6 Hz), 9.27 (d, 1H, J=9.2 Hz). ¹³C NMR (100 M Hz, CDCl₃): 14.2, 39.0, 119.8, 120.5, 123.0, 123.4, 124.7, 125.0, 125.3, 125.4, 125.5, 125.6, 126.5, 125.8, 127.1, 127.2, 127.7, 128.2, 129.0, 129.3, 130.0, 130.1, 131.8, 136.4, 137.7, 140.6, 140.8, 141.8. FIRMS (EI, m/z): calcd for C₃₆H₂₅N, 471.1987. found 471.1987 (M⁺).

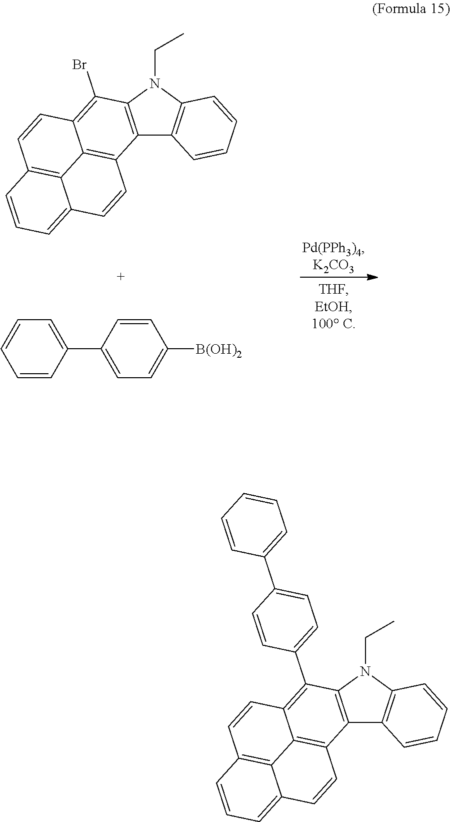 [SMILES: CCn1c2ccccc2c2c3ccc4cccc5ccc(c(Br)c21)c3c54 ; OB(O)c1ccc(-c2ccccc2)cc1 ; CCn1c2ccccc2c2c3ccc4cccc5ccc(c(-c6ccc(-c7ccccc7)cc6)c21)c3c54]

### Example 4

0.4 g of the product of Formula 12 (1.00 mmole), 0.25 g of thiophen-2-ylboronic acid (2.00 mmole), and 3.32 g of potassium carbonate (24.00 mmole) were charged in a one-necked bottle. 12 mL of water, 50 mL of THF, and 10 mL of ethanol were added into the one-necked bottle. The mixture was heated to 60° C. to dissolve the solid, and the bottle was then vacuumed and purged with nitrogen several times. 0.06 g of Pd(PPh₃)₄ (1.00 mmole) was rapidly added into the bottle, and the bottle was then vacuumed and purged with nitrogen several times. The reaction under nitrogen was heated to 100° C. and reacted at 100° C. for two days. The resulting was cooled to room temperature to obtain a crude. The crude was extracted by ethyl acetate and water to collect an organic layer thereof. The organic layer was dried by MgSO₄, and silica gel was then added to the organic layer. The organic layer was then condensed to remove the solvent thereof, and the extracted crude was dry loaded on the silica gel. The extracted crude was purified by chromatography with an eluent of n-hexane to obtain a yellow solid (yield=39.89%). The above reaction is shown as Formula 16. The spectra data of the product of Formula 16 is shown as follows. ¹H NMR (400 M Hz, CDCl₃): 1.18-1.23 (m, 3H), 2.11 (d, 2H, J=28.4 Hz), 7.28-7.36 (m, 2H), 7.43-7.68 (m, 5H), 7.86-7.99 (m, 1H), 8.05 (d, 1H, J=8.0 Hz), 8.15-819 (m, 1H), 826 (d, 1H, J=7.6 Hz), 8.35-8.40 (m, 1H), 8.89 (d, 1H, J=8.4 Hz), 924 (d, 1H, J=9.2 Hz). ¹³C NMR (100 M Hz, CDCl₃): 14.6, 38.9, 109.4, 111.8, 117.6, 119.5, 119.9, 123.0, 123.1, 123.3, 123.4, 124.8, 125.0, 125.1, 125.4, 125.6, 125.7, 127.2, 127.3, 127.4, 128.8 129.6, 129.9, 130.0, 131.2, 137.5, 139.1, 141.7. HRMS (EI, m/z): calcd for C₂₈H₁₉NS 401.1238. found 401.1240 (M⁺).

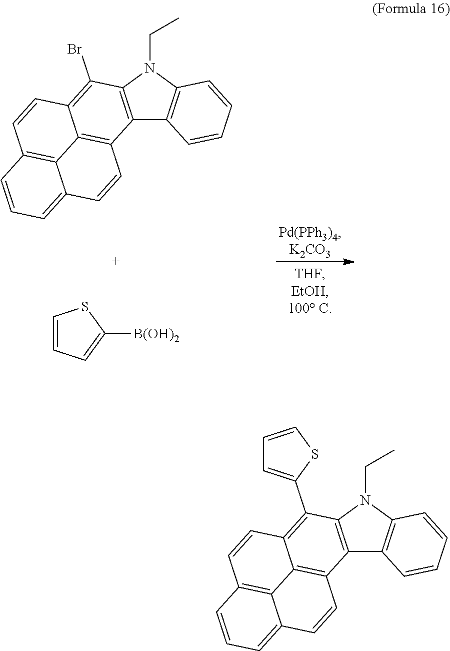 [SMILES: CCn1c2ccccc2c2c3ccc4cccc5ccc(c(Br)c21)c3c54 ; OB(O)c1cccs1 ; CCn1c2ccccc2c2c3ccc4cccc5ccc(c(-c6cccs6)c21)c3c54]

### Example 5

0.29 g of the product of Formula 10 (1.00 mmole), 0.4 mL of 1-bromo-4-tert-butylbenzene (2.00 mmole), and 0.1 g of Pd₂(dba)₃ (0.1 mmole) were charged in a high pressure tube. 0.02 g of P(t-Bu)₃ (0.08 mmole), 0.58 g of sodium tert-butoxide (6.00 mmole) and 2 mL of xylene were weighted and added into the high pressure tube in a dry box. The high pressure tube was then sealed, heated to 140° C., and reacted at 140° C. for 4 days. The resulting was cooled to room temperature and filtered. The filtered cake was washed by THF, and silica gel was added to the filtrate. The filtrate was condensed to remove the solvent thereof, and a crude was dry loaded on the silica gel. The crude was then purified by chromatography with an eluent of n-hexane to obtain a yellow solid (yield=70.88%). The above reaction is shown as Formula 17. The spectra data of the product of Formula 17 is shown as follows. ¹H NMR (400 M Hz, CDCl₃): ¹H NMR (400 M Hz, CDCl₃): 1.49 (s, 9H), 7.48-7.62 (m, 5H), 7.69 (d, 2H, J=8.4 Hz), 7.95-8.02 (m, 3H), 8.13 (s, 1H), 8.17 (d, 1H, J=7.6 Hz), 8.26 (d, 1H, J=7.2 Hz), 8.36 (d, 1H, J=9.2 Hz), 8.87 (d, 1H, J=7.6 Hz), 9.19 (d, 1H, J=9.2 Hz). ¹³C NMR (100 M Hz, CDCl₃): 31.5, 34.9 106.5, 110.7, 117.0, 120.3, 122.7, 123.5, 123.6, 124.6, 125.1, 124.3, 125.4, 125.5, 126.7, 126.9, 127.1, 127.3, 127.8, 128.3, 129.9 130.3, 130.5, 134.8, 139.8, 142.0, 151.0. HRMS (EI, m/z): calcd for C₃₂H₂₅N, 423.1987. found 423.1985 (M⁺). Anal. Calcd. for C₃₂H₂₅N: C, 90.74; H, 5.95; N, 3.31%. Found: C, 90.57; H, 5.95; N, 3.09%.

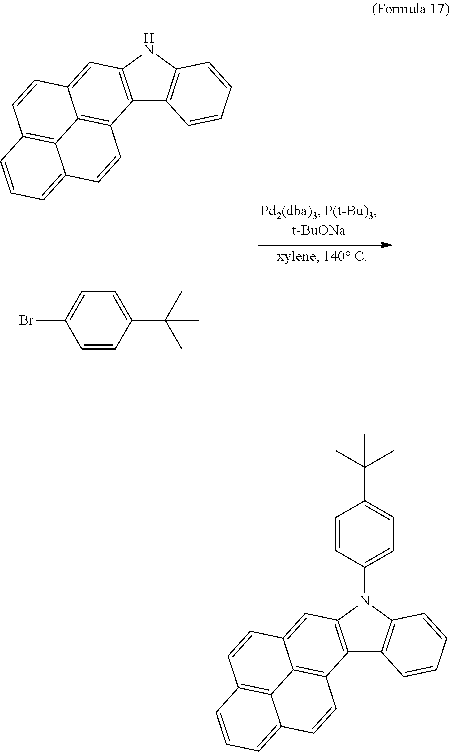 [SMILES: c1cc2ccc3cc4[nH]c5ccccc5c4c4ccc(c1)c2c34 ; CC(C)(C)c1ccc(Br)cc1 ; CC(C)(C)c1ccc(-n2c3ccccc3c3c4ccc5cccc6ccc(cc32)c4c65)cc1]

### Example 6

0.29 g of the product of Formula 10 (1.00 mmole), 0.72 g of 2-(4-bromophenyl)-4-phenylquinoline (2.00 mmole), and 0.1 g of Pd₂(dba)₃ (0.1 mmole) were charged in a high pressure tube. 0.02 g of P(t-Bu)₃ (0.08 mmole), 0.58 g of sodium tert-butoxide (6.00 mmole) and 2 mL of xylene were weighted and added into the high pressure tube in a dry box. The high pressure tube was then sealed, heated to 140° C., and reacted at 140° C. for 4 days. The resulting was cooled to room temperature and filtered. The filtered cake was washed by THF, and silica gel was added to the filtrate. The filtrate was condensed to remove the solvent thereof, and a crude was dry loaded on the silica gel. The crude was then purified by chromatography with an eluent of n-hexane to obtain a yellow solid (yield=87.69%). The above reaction is shown as Formula 18. The spectra data of the product of Formula 18 is shown as follows. ¹H NMR (400 M Hz, CDCl₃): 7.51-7.67 (m, 10H), 7.77-7.81 (m, 1H), 7.82 (d, 1H, J=8.0 Hz), 7.85-8.02 (m, 5H), 8.17-8.20 (m, 2H), 8.26 (d, 1H, J=7.2 Hz), 8.32 (d, 1H, J=8.8 Hz), 8.37 (d, 1H, J=7.2 Hz), 8.51 (d, 2H, J=8.4 Hz), 8.88 (d, 1H, J=7.6 Hz), 9.19 (d, 1H, J=9.2 Hz). ¹³C NMR (100 M Hz, CDCl₃): 106.5, 110.0, 117.2, 119.5, 120.6, 120.7, 122.8, 123.5, 124.0 124.7, 124.2, 125.2, 125.4, 125.5, 125.7, 125.8, 126.0, 126.8, 126.9, 127.2, 127.8, 128.1, 128.5, 128.8, 129.6, 129.9, 130.2, 130.5, 130.6, 139.6, 141.6, 155.5. HRMS (EI, m/z): calcd for C₄₃H₂₆N₂ 570.2096. found 570.2091 (M⁺). Anal. Calcd. for C₄₃H₂₆N₂: C, 90.50; H, 4.59; N, 4.91%. Found: C, 90.48; H, 4.65; N, 4.91%.

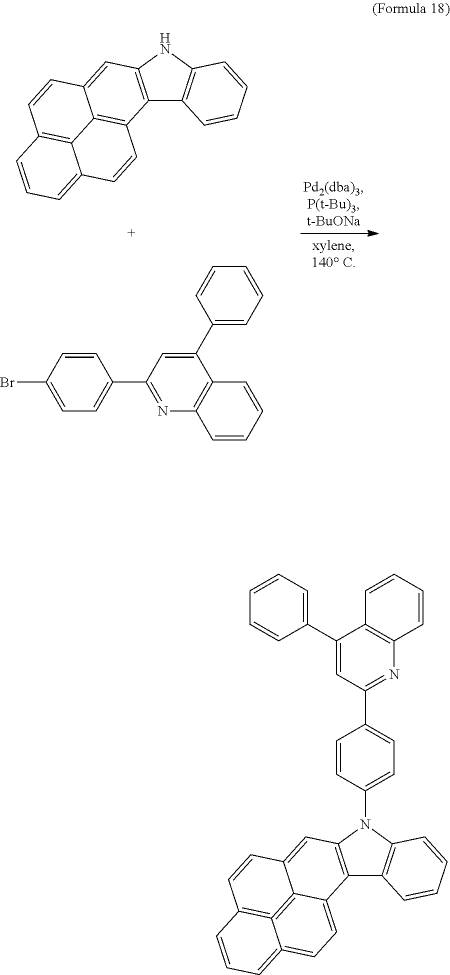

### Example 7

0.29 g of the product of Formula 10 (1.00 mmole), 0.32 g of bromobenzene (2.00 mmole), and 0.1 g of Pd₂(dba)₃ (0.1 mmole) were charged in a high pressure tube. 0.02 g of P(t-Bu)₃ (0.08 mmole), 0.58 g of sodium tert-butoxide (6.00 mmole) and 2 mL of xylene were weighted and added into the high pressure tube in a dry box. The high pressure tube was then sealed, heated to 140° C., and reacted at 140° C. for 4 days. The resulting was cooled to room temperature and filtered. The filtered cake was washed by THF, and silica gel was added to the filtrate. The filtrate was condensed to remove the solvent thereof, and a crude was dry loaded on the silica gel. The crude was then purified by chromatography with an eluent of n-hexane to obtain a yellow solid (yield=85.31%). The above reaction is shown as Formula 19. The spectra data of the product of Formula 19 is shown as follows. ¹H NMR (400 M Hz, CDCl₃): 7.49-7.60 (m, 4H), 7.71 (d, 4H, J=4.0 Hz), 7.96-8.03 (m, 3H), 8.11 (s, 1H), 8.18 (d, 1H, J=7.6 Hz), 8.27 (d, 1H, J=7.6 Hz), 8.39 (d, 1H, J=8.8 Hz), 8.88 (d, 1H, J=7.2 Hz), 9.21 (d, 1H, J=7.6 Hz).

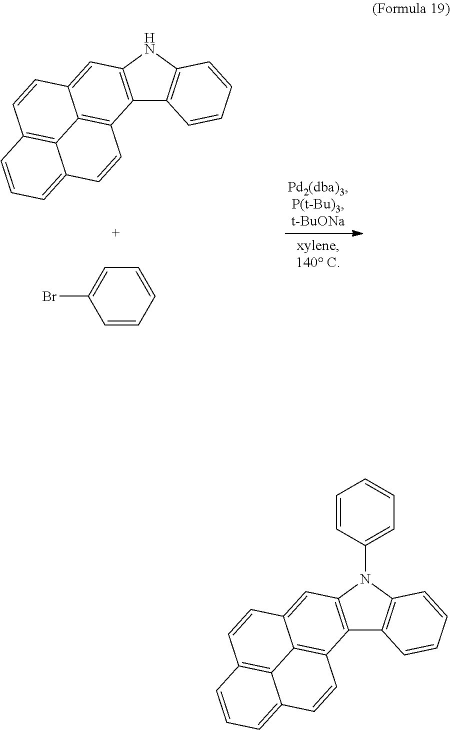 [SMILES: c1cc2ccc3cc4[nH]c5ccccc5c4c4ccc(c1)c2c34 ; Brc1ccccc1 ; c1ccc(-n2c3ccccc3c3c4ccc5cccc6ccc(cc32)c4c65)cc1]

### Example 8

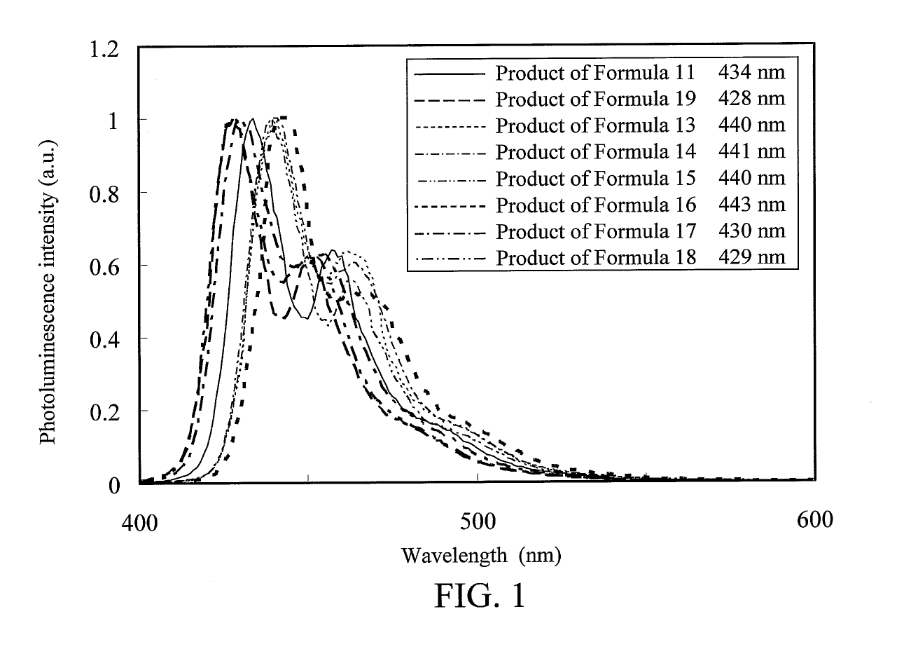

The product of Formula 11 (Preparation Example 3), the product of Formula 13 (Example 1), the product of Formula 14 (Example 2), the product of Formula 15 (Example 3), the product of Formula 19 (Example 7), the product of Formula 17 (Example 5), the product of Formula 18 (Example 6), and the product of Formula 16 (Example 4) were dissolved in dichloromethane, respectively, for preparing 10⁻⁵M solutions to measure their photoluminescence intensities, as shown in FIG. 1. As shown in a photoluminescence intensity comparison between the solutions of the products of Formulae 11, 13, and 14, the different aryl groups (Ar) substituted on the pyrenyl group will increase a conjugation length of the carbazole serial compounds, such that the emission wavelengths of the carbazole serial compounds will slightly red shift. As shown in a photoluminescence intensity comparison between the solutions of the products of Formulae 19, 17, and 18, the different aryl groups substituted on the nitrogen atom will not significantly influence the emission wavelength of the carbazole serial compound.

### Example 9

ITO was served as an anode, 60 nm of NPNPB (Formula 20) served as a hole injecting layer, 10 nm of NPB (Formula 21) served as a hole transporting layer, 3% of the carbazole serial compound served as a guest material and 97% of DMPPP served as a host material of a light emitting layer having a thickness of 30 nm, 20 nm of BAlq (Formula 22) served as a electron transporting layer, 1 nm of LiF served as an electron injecting layer, and Al served as a cathode were sequentially formed on the ITO anode. In another example, the ITO was served as an anode, 50 nm of NPB served as a hole transporting layer, 30 nm of the host material (such as the compound in Formula 16) served as a light emitting layer, 10 nm of the BCP served as a hole blocking layer, 30 nm of Alq₃ served as a electron transporting layer, 55 nm of magnesium silver alloy served as a electron injecting layer, and 100 nm of Ag served as a cathode were sequentially formed on the ITO anode to complete an OLED.

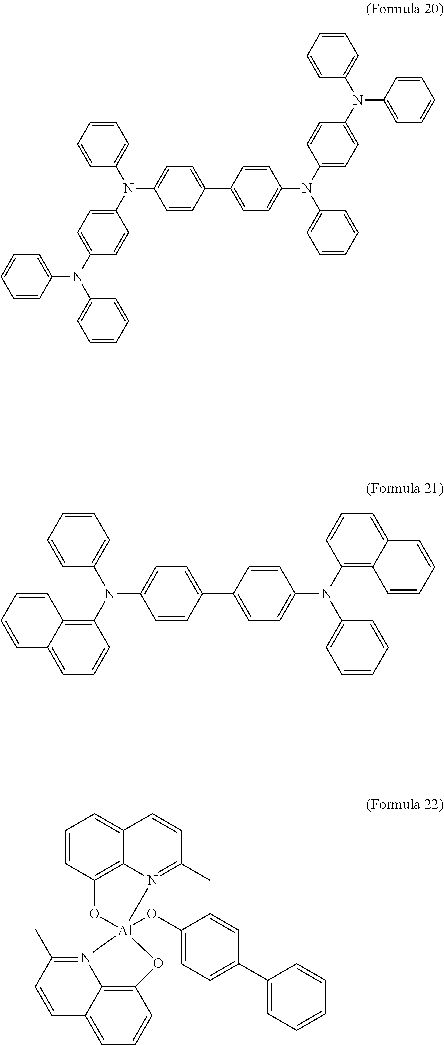

The luminance, the external quantum efficiency (E.Q.E.), the maximum emission wavelength (λₘₐₓ), the full width at half maximum (FWHM), the CIE coordination, and lifetime (T_{0.8}) of the devices are tabulated in Table 1. The brightness and the EQE of the devices were measured at a driving voltage of 6V. The value in parentheses of the λₘₐₓ column means a relative intensity of an emission wavelength. The lifetime (T_{0.8}) of the device is defined as a period from an initial brightness (100%) decayed to 80% brightness of the device operated by a constant current.

TABLE 1

| Host material | L(cd/m²) | EQE (%) | λₘₐₓ (nm) | FWHM(nm) | CIE (x, y) | T_{0.8} (h) |
|---|---|---|---|---|---|---|
| Product of | 270 | 4.63 | 440 (1.00), | 60 | (0.174, 0.174) | 3.1 |
| Formula 11 | | | 467(0.91) | | | |
| Product of | 488 | 4.59 | 443(1.00), | 52 | (0.150, 0.127) | 21 |
| Formula 13 | | | 469 (0.82) | | | |
| Product of | 743 | 4.52 | 443(1.00), | 50 | (0.154, 0.131) | 32 |
| Formula 15 | | | 470(0.77) | | | |
| Product of | 608 | 4.78 | 443(1.00), | 47 | (0.152, 0.123) | 55 |
| Formula 14 | | | 470 (0.73) | | | |
| Product of | 119 | 3.09 | 448(1.00) | 70 | (0.176, 0.173) | 15 |
| Formula 19 | | | | | | |
| Product of | 381 | 3.87 | 436(0.99), | 72 | (0.158, 0.148) | 42 |
| Formula 17 | | | 461 (1.00) | | | |
| Product of | 488 | 4.59 | 434(1.00), | 59 | (0.159, 0.126) | 49 |
| Formula 18 | | | 456 (0.87) | | | |

Compared to the product of Formula 11 without any substituent group on the pyrenyl group, the products of Formulae 13-15 with different Ar (aryl groups) substituted on the pyrenyl group have narrower emission bandwidths (FWHM=52-47 nm) and weaker shoulder intensities. As such, the products of Formulae 13-15 emit deeper blue lights. When the lifetimes of the devices operated at a constant current are compared on the basis of initial brightness of 500 cd/m², the devices utilizing the products of Formulae 13-15 are seven to seventeen times the lifetime of the device utilizing the product of Formula 11. Accordingly, the carbazole serial compounds having an aryl group substituted on the pyrenyl group may efficiently improve device lifetime.

As shown in FIG. 1, the product of Formula 16 has a blue emission wavelength of 450 nm to 460 nm, which is the sensitive region of human eyes. Accordingly, the specific aryl group substituted on the pyrenyl group may tune the emission spectra of the carbazole serial compound.

In addition, the devices utilizing the products of Formulae 17-18 are about 1.25 to 1.5 times the EQE of the device utilizing the product of Formula 19. The devices utilizing the products of Formulae 17-18 are 3 times the lifetime of the device utilizing the product of Formula 19. The device utilizing the product of Formula 18 has a narrower FWHM than the device utilizing the product of Formula 19. Accordingly, X substituted on the para position of the phenyl group substituted on nitrogen of the carbazole serial compound may improve device performance.

While the disclosure has been described by way of example and in terms of the preferred embodiments, it is to be understood that the disclosure is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A carbazole serial compound, having a formula: 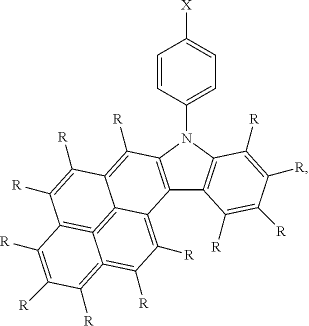
wherein X is selected from a halogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group, and
wherein each R is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

2. The carbazole serial compound as claimed in claim 1, having a formula:

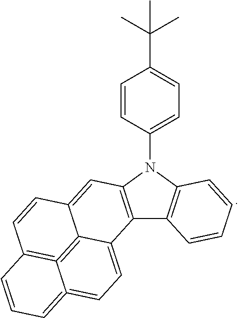

3. The carbazole serial compound as claimed in claim 1, having a formula:

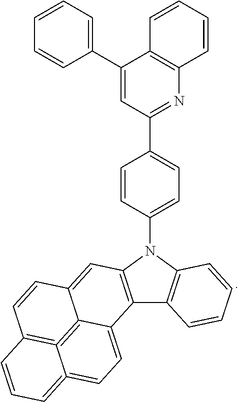

4. A carbazole serial compound, having a formula: 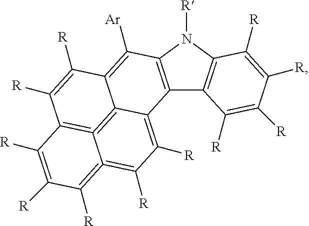
wherein Ar is selected from a para-t-butyl phenyl group, a biphenyl group, a naphthalenyl group, or a thienyl group,
wherein R′ is selected from a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, or a substituted or non-substituted C₄₋₄₀ hetero aryl group, and
wherein each R is independently selected from a hydrogen atom, a cyano group, a substituted or non-substituted C₁₋₄₀ alkyl group, a substituted or non-substituted C₂₋₄₀ alkenyl group, a substituted or non-substituted C₂₋₄₀ alkynyl group, a substituted or non-substituted C₆₋₄₀ aryl group, a substituted or non-substituted C₄₋₄₀ hetero aryl group, a substituted or non-substituted C₆₋₄₀ aryl amino group, or a substituted or non-substituted C₁₋₄₀ alkyl amino group.

5. The carbazole serial compound as claimed in claim 4, having a formula:

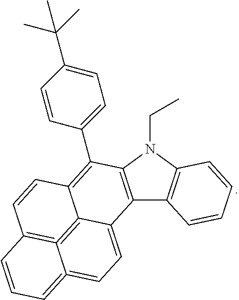

6. The carbazole serial compound as claimed in claim 4, having a formula:

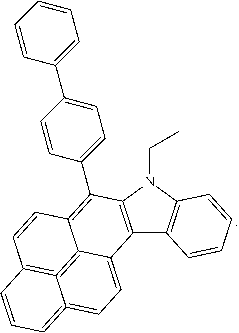

7. The carbazole serial compound as claimed in claim 4, having a formula:

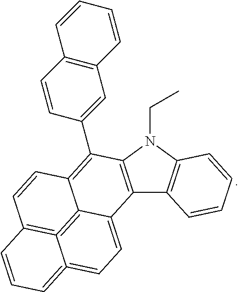

8. The carbazole serial compound as claimed in claim 4, having a formula:

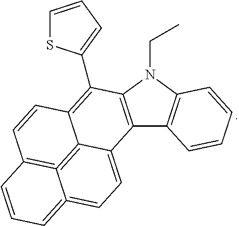

9. An organic light emitting diode, comprising:
an anode;
a cathode; and
an organic layer disposed between the anode and the cathode,
wherein the organic layer comprises the carbazole serial compound as claimed in claim 1.
